(19) United States
(12) Patent Application Publication
(10) Pub. No.: US 2004/0132672 A1
(43) Pub. Date: Jul. 8, 2004

(54) Method for inhibiting cancer cells

(75) Inventors: Muraleedharan G. Nair, Okemos, MI (US); Yanjun Zhang, East Lansing, MI (US); Shaiju K. Vareed, East Lansing, MI (US)

Correspondence Address: MCLEOD & MOYNE, P.C., 2190 COMMONS PARKWAY, OKEMOS, MI, 48864, US

(73) Assignee: Board of Trustees of Michigan State University, East Lansing, MI

(21) Appl. No.: 10/725,214

(22) Filed: Dec. 1, 2003

Related U.S. Application Data
(63) Continuation-in-part of application No. 09/776,527, filed on Feb. 2, 2001, now Pat. No. 6,656,914.
Application No. 09/776,527 is a continuation of application No. 09/494,077, filed on Jan. 28, 2000, now abandoned.

(51) Int. Cl. A61K 31/7048; A61K 31/353

(52) U.S. Cl. 514/27; 514/456

(57) ABSTRACT

A method for inhibiting stomach or colon cancer cells in a mammal using a composition comprising an anthocyanidin. The method involves using the composition in an amount and for a time to inhibit the cancer cells. The composition can include other anticancer agents.

### CROSS REFERENCE TO RELATED APPLICATIONS

This application is a continuation-in-part application of U.S. Ser. No. 09/776,527, filed Feb. 2, 2001, which is a continuation of U.S. Ser. No. 09/494,077, filed Jan. 28, 2000, now abandoned.

### BACKGROUND OF THE INVENTION

(1) Field of the Invention

The present invention relates to a method for inhibiting cancer cells preferably by feeding a mammal a composition comprising anthocyanidins selected from the group consisting of cyanidin, delphinidin, malvidin, pelargonidin, petunidin and mixtures thereof. In particular, the compositions of anthocyanins inhibit the proliferation of stomach or colon cancer cells.

(2) Description of Related Art

Tumors occur in mammals and can be life threatening. In humans this can include prostate, colon, breast, lung, and kidney, prostate, liver, lymphoma/CNS, leukemia, pancreatic, gastric, esophageal, ovarian, uterine and testicular tumors, for instance.

Colon cancer is the second most common cause of cancer mortality and the fourth most common in incidence in the United States (American Cancer Society, Cancer Facts and Figures 1997). Diet has been considered to account for 30% of incidence of colon cancer (Doll and Peto, J. Natl Cancer Inst 66:1192-1308 (1981)). Epidemiological studies have shown consuming fruits and vegetables lowers incidences of various cancers including colon cancer. This anticancer effect of fruits and vegetables is thought to be due in part to antioxidant effects of phytochemicals (Stavric, B., Clin Biochem 27:319-332 (1994)). Other potential anticancer mechanisms are inhibition of carcinogen formation, blocking biotransforming enzyme actions, inducing oxidative detoxification, and trapping and scavenging electrophilic agents (Stavric, B., Clin Biochem 27:319-332 (1994)).

Tart cherries contain various phytochemicals including-anthocyanins and cyanidin. Anthocyanins are flavonoid pigments in many fruits and vegetables as well as cherries. Cyanidin is the major aglycone in cherries and its glycosylated form provides the anthocyanins. All anthocyanins>are derivatives of the basic flavylium cation structure. Montmorency and Balaton cherries contain 120 and 220 mg/g, respectively, of anthocyanins (Wang, H., et al, J. Nat Prod 62:86-88 (1999)). These anthocyanins have been found to be antioxidants of lipids, particularly in foods as described in U.S. Pat. No. 5,985,636 to Gray et al., and inhibit cyclooxygenase enzymes as described in U.S. Pat. No. 6,194,469. Cyanidin was intermediate in efficacy between aspirin and the non-steroidal anti-inflammatory drug, flurbiprofen. The anthocyanins are labile to heating and drying destroys their effectiveness.

The Min mouse has been proposed to be a model for the study of human colorectal cancer (Moser, A. R., et al, Science 247:322-324 (1990)). A mutant mouse lineage predisposed to multiple intestinal neoplasia (Min) results from a mutation in the murine homolog of the adenomatous polyposis coli (APC) gene (Su, L. K., et al, science 256:668-670 (1992)). The APC gene is also mutated in humans who develop sporadic colon cancer as well as persons with familial adenomatosis polyposis (FAP), an autosomal dominantly inherited disease that predisposes to colorectal cancer. The primary phenotype of mice carrying this mutation appears to be the development of multiple adenomas, which progress to adenocarcinomas of the intestine in older mice. Min is transmitted by affected mice to 50% of progeny with an unbiased sex distribution, as is characteristic of a fully penetrant autosomal dominant trait (Moser, A. R., et al, Science 247:322-324 (1990)). The Min mouse strain is an excellent animal model for the anticarcinogenic potential of dietary factors and other potential cancer therapeutic agents (e.g. NSAIDS).

Non-steroidal anti-inflammatory drugs (NSAIDs) that inhibit cyclooxygenase (COX) enzymes have been found to possess preventive effects for colon cancer. Research on the NSAIDs sulindac and proxicam in Min mice showed that they reduced the incidence of intestinal tumors (Boolbol, S. K., et al, Cancer Res. 56:2556-2560 (1996); Jacoby, R. F., et al, Cancer Res. 56:710-714 (1996)). There is a need for a method of treatment which does not involve NSAIDS and is based upon a phytoceutical.

U.S. Pat. No. 5,925,620 to Ohlenschläger et al disclose the use of anthocyanidins with reduced glutathione for the treatment of various diseases. There is no description of the treatment of colon or stomach cancers.

### SUMMARY OF THE INVENTION

The present invention relates to a method for the inhibition of proliferation of cancer cells of the stomach or colon which comprises:

providing with the cells an effective amount of anthocyanidin selected from the group consisting of cyanidin, delphinidin, malvidin, pelargonidin, petunidin and mixtures thereof, in order to inhibit the proliferation of the cells.

Anthocyanins are flavonoid pigments in blue and red fruits and vegetables. Cyanidin for instance is the primary aglycone form of tart cherry anthocyanin. The other anthocyanidins are cyanidin, delphinidin, malvidin, pelargonidin, petunidin and mixtures thereof and sources of these compounds are well known. The dosage amount is preferably between about 0.1 and 300 mg per day per kg of body weight of the mammal.

Preferably the anthocyanidins are between about 70% to 100% by weight of the composition, with the balance, if present, being the anthocyanins, phenolics and the bioflavonoids. U.S. Pat. No. 5,985,636 to Gray et al describes the isolation of the anthocyanins in detail.

The compositions of the present invention can be combined with other active agents which have antitumor properties to provide greater effectiveness. These include NSAIDS.

The term “cancer cells” includes “tumors” as a collection of cells and other cells which undergo unregulated growth.

The term “inhibiting” means preventing the formation of the cancer cells or tumors and/or causing the cancer cells or tumors to shrink. The term “tumor” includes carcinomas, sarcomas and lymphoid tumors.

The term “anthocyanins” means the compounds that impart color in berries.

The term “anthocyanidin” refers to the aglycones of the anthocyanins.

The term “phenolics” refers to compounds with a phenyl group and having one or more hydroxyl groups from berries.

The compounds of the present invention can be applied topically or can be fed orally depending upon the type of tumor or cancer cells. Enteral administration can be via nasogastric tube or percutaneous enterogastrostomy (PEG). Parenteral administration can be by administration (peripheral or central). They can also be injected into the tumor. In each instance a suitable carrier and an adjuvant is included where necessary.

### OBJECTS

It is therefore an object of the present invention to provide a natural source anthocyanidin composition which can be used as an anticancer agent. It is further an object of the present invention to provide naturally a occurring phytoceutical which is inexpensive to prepare. These and other objects will become increasingly apparent by reference to the following description and the drawings.

### BRIEF DESCRIPTION OF THE DRAWINGS

FIG. 1 shows the structure of select anthocyanins (colorants) that have been isolated from BALATON and MONTMORENCY cherries. The aglycone cyanidin has a hydroxyl group at position 3.

FIG. 2 shows the structure of the anthocyanidins.

FIGS. 3 to 7 are graphs of cell viability vs. concentration in ppm when treated in vitro with individual isolated anthocyanidins.

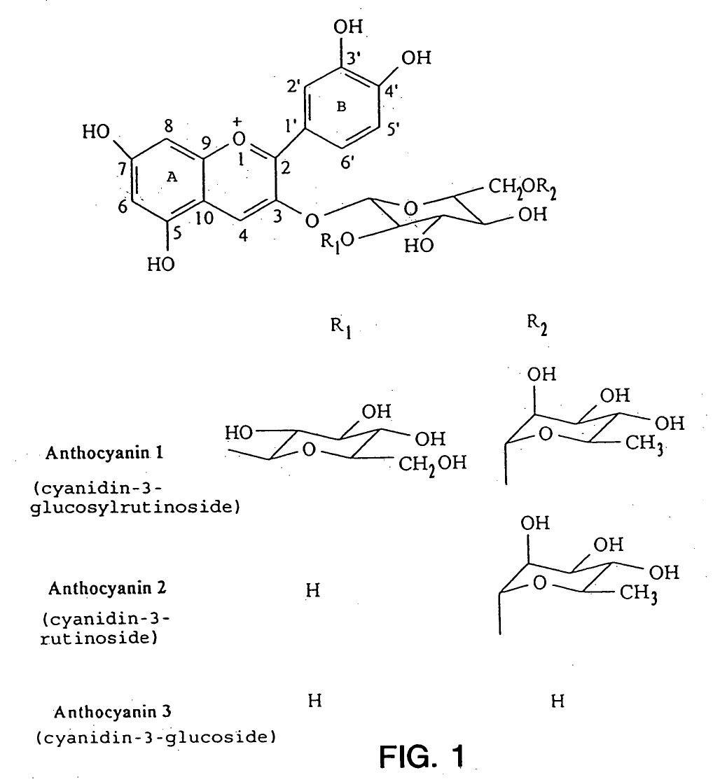

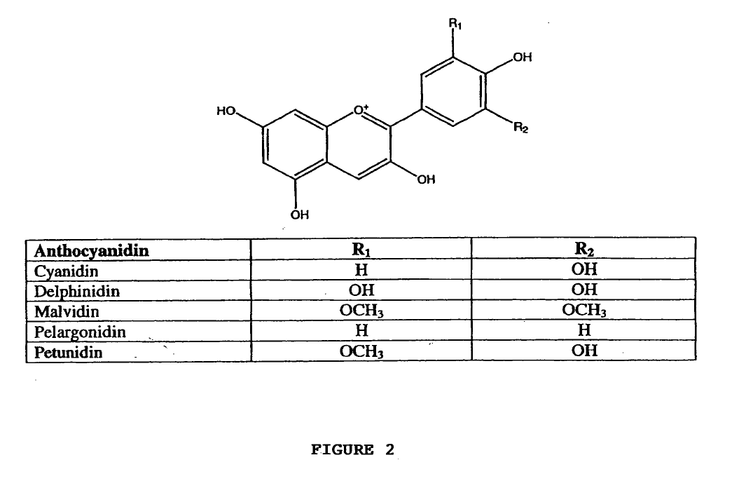

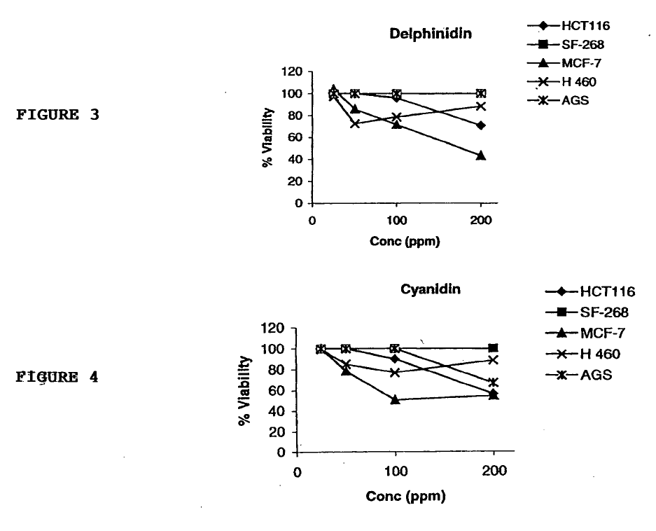

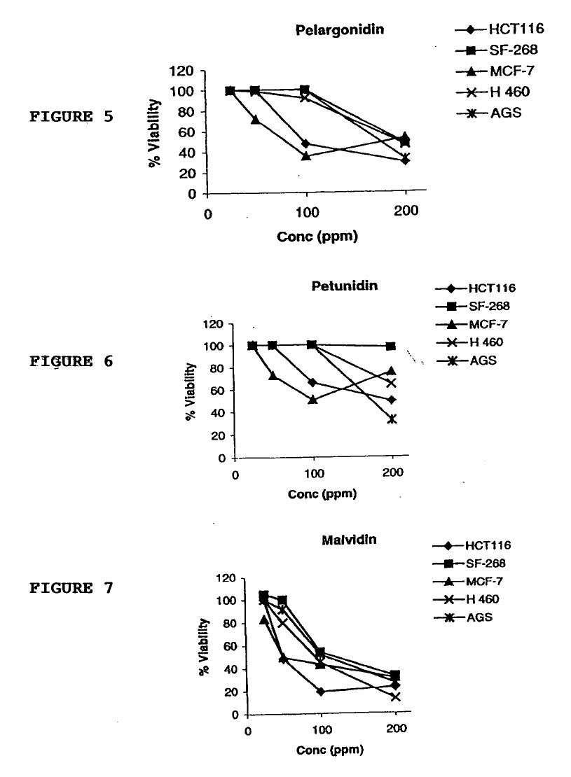

### DESCRIPTION OF PREFERRED EMBODIMENTS

A preferred consumable composition for use in the method comprises in admixture: dried mixture of isolated an anthocyanidin, a food grade carrier, wherein the weight ratio of the mixture to the carrier is between about 0.1 to 100 and 100 to 0.1.

A preferred method is provided for inhibiting stomach or colon tumors or cancer cells in a mammal which comprises feeding the mammal a consumable composition which comprises in admixture: dried mixture of isolated as anthocyanidin; and a food grade carrier wherein the weight ratio of the mixture to the carrier is between about 0.1 to 100 and 100 to 0.1.

The isolated anthocyanidin can be used as a natural nutraceutical/dietary supplement. In this regard, the isolated compound can be provided in a powdered, liquid, or solid form. For example, the mixture may be in a reconstitutable powder composition that, when reconstituted with, for example, water, milk or some other similar liquid will provide a drink. Alternatively, the mixture may be in a solid form such as tablets, gel caps, soft gels, and the like. In addition, the mixture may be incorporated into foodstuffs. In general, a mixture may be provided in a form such that the anthocyanidin is present in an amount in the range from about 0.01% to about 50%, preferably from about 0.1% to about 30%, more preferably, from about 0.5% to about 25%, by weight of the total composition. As an example, when the mixtures are provided in the form of a tablet, the tablet may provide a daily dose of the anthocyanidin of about 0.1 mg to 300 mg, desirably from 1 to 200 mg, preferably a daily dose of 60-100 mg.

A preferred method for producing a mixture comprising anthocyanins, bioflavonoids and phenolics from berries as a composition comprises providing an aqueous solution containing the anthocyanins, bioflavonoids and phenolics from the berries; removing the anthocyanins, bioflavonoids and phenolics onto a resin surface from the aqueous solution; eluting the resin surface with an eluant to remove the anthocyanins, bioflavonoids from the resin surface; and separating the eluant from the anthocyanins, bioflavonoids and then departing the anthocyanidins with hydrolysis of the anthocyanins which are glycosolated.

The resin has a surface to which the anthocyanins and bioflavonoids are adsorbed. A preferred class of adsorptive resins are polymeric crosslinked resins composed of styrene and divinylbenzene such as, for example, the AMBERLITE series of resins, e.g., AMBERLITE XAD-4 and AMBERLITE XAD-16, which are available commercially from Rohm & Haas Co., Philadelphia, Pa. Other polymeric crosslinked styrene and divinylbenzene adsorptive resins suitable for use according to the invention are XFS-4257, XFS-4022, XUS-40323 and XUS-40322 manufactured by The Dow Chemical Company, Midland, Mich., and the like.

It is preferred to use commercially available, government-approved (where required), styrene-divinyl-benzene (SDVB) cross-linked copolymer resin, (e.g., AMBERLITE XAD-16). Thus, in the preferred embodiment, AMBERLITE XAD-16, commercially available from Rohm and Haas Company, and described in U.S. Pat. No. 4,297,220, is used as the resin. This resin is a non-ionic hydrophobic, cross-linked polystyrene divinyl benzene adsorbent resin. AMBERLITE XAD-16 has a macroreticular structure, with both a continuous polymer phase and a continuous pore phase. In a particularly preferred embodiment, the resin used in the present invention has a particle size ranging from 100-200 microns.

It is contemplated that other adsorbents such as those in the AMBERLITE XAD adsorbent series, which contain hydrophobic macroreticular resin beads, with particle sizes in the range of 100-200 microns, will also be effective in the methods of the present invention. Moreover, different variations of the AMBERLITES, such as the AMERCHRON CG series of adsorbents, used with particle sizes in the range of 100-200 microns, may also be suitable for use, in the present invention. The AMBERLITE XAD-16 is preferred since it can be re-used many times (over 100 times) However, it is contemplated that for food, the use of governmentally-approved resins in the present invention will be considered important and/or desirable.

Any solvent can be used to remove the adsorbed anthocyanins, bioflavonoids and phenolics. Preferred are lower alkanols containing 1 to 4 carbon atoms and most preferred is ethanol (ethyl alcohol) since it is approved for food use. Typically the ethanol is azeotroped with water; however, absolute ethanol can be used. Water containing malic acid and sugars in the cherries pass through the column. These are collected and can be used in foods as flavors.

The anthocyanidins are commercially available and can be isolated from fruits and vegetables. The cyanidins can be isolated from the BALATON and the MONTMORENCY cherries and hydrolyzed to cyanidin for instance. The composition of the cherries is in part shown by U.S. Pat. No. 5,985,636 and in part U.S. Pat. No. 6,150,408, which are incorporated by reference herein. Parent application Ser. No. 09/776,527 is also incorporated by reference.

The term “carrier” or “bulking agent” is used to mean a composition, which is added to increase the volume of the composition of the purified composition. The bulking agent can include any edible starch containing material, protein, such as non-fat dry milk. Within this group are flour, sugar, soybean meal, maltodextrin and various condiments, such as salt, pepper, spices and herbs, for instance. The bulking agent is used in an amount between about 10⁻⁶ and 10⁶ parts by weight of the mixture.

The composition of the anthocyanidin(s) is introduced into the food in an amount between about 0.1 and 300 mg/gm of the active ingredients per gram of the food. The amount is preferably selected so as to not affect the taste of the food and to produce the most beneficial result. The food can be high (wet) or low moisture (dry) as is well known to those skilled in the art. When used as a dietary supplement the tablets contain between 0.1 to 1 gram of active ingredient. A particular food is cooked meat and other prepared foods where the composition provide antioxidant properties to the food and optionally color. The composition can be dispensed as a condiment on the prepared food.

Methods have been developed for extraction and isolation of phytochemicals are well known in the art and are described by Chandra, A. et al., J. Agric. Food Chem. 41:1062 (1992); Wang, H., et al., J. Agric. Food Chem. 45:2556-2560 (1997). A method for rapid screening of antioxidant activity (Arora, A. and G. M. Strasburg, J. Amer. Oil Chem. [symbol] (1997)).

The following Examples 1 to 5 show that tart cherry anthocyanins, cyanidin, or cherry fruits inhibit intestinal tumorigenesis in Min mice. Forty-eight Min mice were randomly assigned to five treatment groups at 4-5 weeks of age and fed treatment diets for 10 weeks. The treatments were:

1) Modified AIN-93G control diet, 2) The control diet+800 ppm anthocyanins in drinking water, 3) The control diet+200 ppm cyanidin in drinking water, 4) The control diet+200 ppm sulindac in drinking water, 5) Modified control diet containing 20% freeze dried pitted tart cherries. Only mean diameter, not the number of adenomas in the small intestine was reduced by sulindac, whereas diameter was increased by cherry diet (p<0.05). Mice consuming cherry diet, anthocyanins, or cyanidin had significantly fewer cecal adenomas than the controls, whereas mice consuming sulindac had significantly more cecal adenomas than controls. Mice treated with sulindac had the greatest number of colonic adenomas (p<0.05). Colon tumor volume was not significantly influenced by treatment. Sulindac inhibits small intestinal tumorigenesis and anthocyanins and cyanidin inhibit cecal tumorigenesis. This suggests that they may have different target sites in the intestine for exerting their antitumorigenic actions in Min mice.

Methods

All research was conducted with approval of the Michigan State University, East Lansing, Michigan, All-University Committee on animal use and care. Mice were housed in MSU Laboratory, Animal Resources maintained facilities. A colony of Min mice was maintained by crossing male Min mice (Apc^{min}/Apc⁺) with normal adult C57BL/6J female mice. Mice were housed in a temperature and humidity-controlled room (20-220C, 70%) with a 12-h light/dark cycle. At three weeks of age, mice were bled from the dorsal pedal vein (30 μL) for genotyping analysis to identify Apc^{min}/Apc⁺ using polymerase chain reaction (PCR) analysis and subsequent gel electrophoresis analysis. Forty-eight Min mice identified were randomly assigned to five treatment groups (7 to 11 per treatment) at 4 or 5 weeks of age and fed treatment diets for 10 weeks. The treatments were:

1) Modified AIN-93G control diet and 50 ppm ascorbic acid in drinking water (n=11)

2) The control diet+50 ppm ascorbic acid and 800 ppm anthocyanins in drinking water (n=9)

3) The control diet+50 ppm ascorbic acid and 200 ppm cyanidin in drinking water (n=7)

4) The control diet+50 ppm ascorbic acid and 200 ppm sulindac in drinking water (n=10)

5) 20% freeze-dried cherries+50 ppm ascorbic acid in drinking water (n=11).

Ingredient composition of diets is in Table 1. All diets contained 22% protein, 15% fat (soybean oil) and 5% cellulose contents. Distilled water was used for drinking water. Ascorbic acid was added to provide low pH for keeping anthocyanins and cyanidin in solution since they are stable only under pH 7. The concentration of sulindac (200 ppm) was based on the effective range found from most studies that have shown sulindac to reduce intestinal neoplasia. Cyanidin concentration (200 ppm) was matched to that of sulindac. Anthocyanins was tested at the level four times the cyanidin concentration because anthocyanins are the glycosylated cyanidin and the level (800 ppm) has equivalent amount of flavylium cation. Red tart pitted cherries (Peerson Farms, Inc., Shelby, Mich.) were frozen, freeze-dried, ground using plate grinder, and then screened to pass a 1 mm screen before they were incorporated into the diet at the expense of sucrose, cornstarch and dyetrose. One hundred grams of the experimental diet included 23 g of ground cherries to make 20% of cherries in the diet because dry matter of cherries was 75%, whereas that of AIN-93G diet was 91%.

Body weight was measured once a week until mice were sacrificed at the end of treatment period. Upon sacrifice by carbon dioxide asphyxiation, the liver was removed and frozen immediately for confirmatory PCR analysis. The entire small intestine, cecum, and colon were removed from each mouse to determine the number and size of adenomas. The tissues were separated into the following sections: proximal one-third of small intestine, middle one-third of small intestine, distal one-third of small intestine, cecum, and colon. All intestinal sections were opened longitudinally, rinsed thoroughly with water, fixed overnight in 10% neutral-buffered formalin, and then stained with 0.2% methylene blue. Tumor number and size (diameter for flat tumors or volume in the case of three-dimensional tumors) were determined in each intestinal segment on 1 mm grid transparency by direct counting with the aid of a dissecting microscope. Tumor numbers in each small intestinal segment were summed to obtain a total small intestine tumor burden for each mouse.

Tumor number and tumor diameter in the small intestine were analyzed by one-way analysis of variance to detect the effects of treatments. For tumor numbers and volume in cecum and colon, data were transformed to ranks and then analyzed by one-way analysis of variance. When significant treatment effects were detected (P<0.05), means were compared using the Least Significant Difference method.

TABLE 1
Composition of the experimental diets
(Percentage of diet)

| | Modified | 20% |
|---|---|---|
| | AIN-93G | Cherries |
| Ingredient | diet | diet |
| Casein | 22.12 | 22.12 |
| Soybean Oil | 15.00 | 15.00 |
| Corn Starch | 31.72 | 24.22 |
| Dyetrose | 10.57 | 8.07 |
| Sucrose | 10.00 | 0.00 |
| AIN-93G-MX | 3.87 | 3.87 |
| AIN-93G-VX | 1.11 | 1.11 |
| L-Cystine | 0.33 | 0.33 |
| Choline Bitartrate | 0.28 | 0.28 |
| Tert-Butylhydroquinone | 0.003 | 0.003 |
| Cellulose | 5.00 | 5.00 |
| Freeze-Dried Cherries | 0.00 | 20.00 |

Results and Discussion

There are no differences found in numbers of adenomas in the small intestine and in the three sections of the small intestine (Table 2). There was a trend that sulindac in drinking water and 20% cherry diet reduced the number of adenoma in the proximal section of the small intestine (P=0.05). These findings are contradicted by the results from many studies which showed significant reduction of intestinal tumor multiplicity by sulindac in Min mice (Mahmoud, N. N., et al, Carcinogenesis 19:87-91 (1998); Chiu, C. -H., et al, Cancer Res. 57:4267-4273 (1997)). Small number of mice per treatment group (7 to 11) and wide range of intrastrain variations on adenoma development may account for these contradictory results. However, mean diameter of adenoma in the small intestine was significantly reduced by sulindac compared to control diet whereas it was increased by cherry diet (Table 3). Neither anthocyanins nor cyanidin in drinking water affected the number and size of the small intestinal adenomas.

TABLE 2
Adenoma numbers in the small intestine

| Treatment | Total | Proximal | Middle | Distal |
|---|---|---|---|---|
| AIN93G Control | 45.9 ± 12.6 | 11.1 ± 2.8 | 15.5 ± 4.7 | 19.3 ± 6.2 |
| Anthocyanins | 67.3 ± 13.9 | 17.0 ± 3.1 | 23.4 ± 5.1 | 26.8 ± 6.8 |
| Cyanidin | 51.3 ± 15.8 | 15.6 ± 3.5 | 15.7 ± 5.8 | 20.0 ± 7.8 |
| Sulindac | 24.7 ± 13.2 | 5.6 ± 2.9 | 9.1 ± 4.9 | 10.0 ± 6.5 |
| Cherries | 37.8 ± 12.6 | 7.8 ± 2.8 | 12.6 ± 4.7 | 17.4 ± 6.2 |

Each value represents mean ± SEM

TABLE 3
Adenoma diameters in the small intestine

| | Total Adenoma Diameter | Mean Adenoma Diameter |
|---|---|---|
| Treatment | (mm) | (mm) |
| AIN93G Control | 65.3 ± 17.6 | 1.39 ± 0.10^{b} |
| Anthocyanins | 90.9 ± 19.4 | 1.34 ± 0.10^{b} |
| Cyanidin | 67.4 ± 22.0 | 1.25 ± 0.11^{b} |
| Sulindac | 23.5 ± 18.4 | 0.93 ± 0.10^{a} |
| Cherries | 67.7 ± 17.6 | 1.66 ± 0.09^{c} |

Each value represents mean ± SEM
Different superscripts indicate significant differences (P < 0.05)

Mice consuming 20% tart cherry diet had less adenomas in the cecum and so did those consuming anthocyanins and cyanidins. In contrast, mice consuming sulindac had a significantly higher number of cecal adenomas. A similar trend was found in the number of adenomas in the colon; cherry and anthocyanin consuming mice had adenomas than mice consuming sulindac. Sulindac consuming mice had twice as many adenomas as those of mice in cherry diet (P<0.05). The size of adenoma in the cecum is determined by their volume. Cherries, anthocyanins and cyanidin reduced, while sulindac increased, the size of adenomas. Cherry was intermediate in efficacy of reduction of the adenoma diameter. No differences were found in the size of the colonic adenomas determined by the three-dimensional volume of adenomas.

TABLE 4
Adenoma numbers and volume in the cecum and colon

| | | Cecal Total | | Colon Total |
|---|---|---|---|---|
| | Cecum | Volume | | Volume |
| Treatment | Number | (mm³) | Colon Number | (mm³) |
| AIN93G | 1.91 ± 0.50^{a} | 2.50 ± 0.77^{a} | 3.00 ± 0.64^{ab} | 1.95 ± 1.79 |
| Control | | | | |
| Antho- | 0.56 ± 0.56^{b} | 0.67 ± 0.85^{b} | 2.78 ± 0.71^{a} | 3.52 ± 1.98 |
| cyanins | | | | |
| Cyanidin | 0.57 ± 0.63^{b} | 0.56 ± 0.96^{b} | 3.71 ± 0.80^{ab} | 3.51 ± 2.24 |
| Sulindac | 4.00 ± 0.53^{c} | 4.00 ± 0.81^{a} | 5.30 ± 0.67^{b} | 3.35 ± 1.88 |
| Cherries | 0.54 ± 0.50^{b} | 1.63 ± 0.77^{b} | 2.36 ± 0.64^{a} | 7.58 ± 1.79 |

Each value represents mean ± SEM
Different superscripts indicate significant differences (P < 0.05)

Solid tumor numbers cecum and colon were determined (tumor was three-dimensional and visibly raised towards the lumenal side of the tissue). There were no differences detected in the average number of solid tumors in the cecum and colon even though anthocyanins and cyanidin treatments numerically reduced the average number (Table 5). In summary, feeding of tart cherry diet (20%) seemed to suppress adenoma multiplicity in cecum and in colon, to a lesser extent. However, feeding cherries enhanced the growth of adenoma in the small intestine by increasing the diameter of adenoma. In the case of sulindac, feeding via drinking water, it significantly reduced the size of adenomas in small intestine but increased the number of adenomas in cecum and colon in Min mice. The chemopreventive effects of anthocyanins, cyanidin and cherry diet and sulindac were not consistent through the intestinal tract suggesting that they may have different target sites in the intestine for exerting their antitumorigenic actions on the development of intestinal neoplasia in Min mice.

TABLE 5
Average number of solid tumors in cecum and colon

| Treatment | Cecum | Colon | Total |
|---|---|---|---|
| AIN93G Control | 0.27 ± 0.11 | 0.27 ± 0.17 | 0.55 ± 0.20 |
| Anthocyanins | 0.00 ± 0.11 | 0.22 ± 0.18 | 0.22 ± 0.22 |
| Cyanidin | 0.00 ± 0.13 | 0.14 ± 0.21 | 0.14 ± 0.25 |
| Sulindac | 0.20 ± 0.11 | 0.20 ± 0.17 | 0.40 ± 0.21 |
| Cherries | 0.18 ± 0.11 | 0.45 ± 0.17 | 0.64 ± 0.20 |

Each value represents mean ± SEM

### EXAMPLES 6 TO 10

The following Examples show the activity of anthocyanidins other than cyanidin. Malvidin and pelargonidin were in particular found to be excellent inhibitors of stomach and colon cancer cell lines in vitro. Stomach cancer has no treatment at the moment. It is significant that these compounds are present in fruits and are very non-toxic. The anthocyanidins with high potency mentioned above are present in red grapes and other fruits.

Cell lines tested: Breast (MCF-7), CNS(SF-268) and lung (NCI-H460) cultures were purchased from the National Cancer Institute (Bethesda, Md.). Colon (HCT-116) and stomach (AGS) cell cultures were purchased from the American Type Culture Collection (Rockville, Md.). Cell cultures are maintained in liquid nitrogen prior to sub-culturing for the assay. For the assay, cell cultures were maintained in an incubator at 37° C. with 5% CO₂ and 80% RH in RPMI-1640 medium supplemented with 10% fetal bovine serum, penicillin (1 unit/100 mL), and streptomycin (1 μg/100 mL). The tumor cell lines were sub-cultured according to their individual growth profiles in order to ensure exponential growth throughout the experiments.

MTT cell proliferation assay: The cells were counted and transferred to 96 well microtiter plates, and incubated for 24 h prior to the addition of test compounds. The cell numbers used for each cancer cell lines were 6000, 3000, 4000, 3000 and 5000 per well for SF-268, NCI H460, MCF-7, HCT-116, and AGS, respectively. Test compounds were dissolved in DSO and diluted with sterile RPMI-1640 media as necessary to obtain the appropriate concentration. The test solutions were then added to the wells containing cells in 100-μL aliquots to obtain final appropriate concentrations. The final concentration of DMSO in each well was 0.2%. Test compounds, positive control, and blank control (DMSO in media) were incubated with all five cell-lines for 48 h, after which MTT solution (5 mg/mL in PBS solution) was added into each well in 25-μL aliquots. The plates then were wrapped in aluminum foil and incubated for three hours at 37° C. with 5% CO₂ and 80% RH. The RPMI media, MTT and floating cells from each well were removed and aliquots of DMSO (20 μL) added into each sample well to dissolve the purple formazan crystals. The plates were then shaken for eight minutes on a gyrorotary shaker after which the absorbence of the contents of each well was recorded with an automated microplate reader (model EL800, Bio-Tek Instruments, Inc., Winooski, Vt.) at 570 nm. The experiments were performed in triplicate at concentrations of 25, 50, 100 and 200 μg/mL. Cell viability was determined by comparing the average absorbance of three test wells verses that of the blank control wells. Results are expressed in a line graph as the percentage of cell viability against concentration of compounds in FIGS. 6 to 10.

It is intended that the foregoing description be only illustrative of the present invention and that the present invention be limited only by the hereinafter appended claims.

We claim:

1. A method for the inhibition of proliferation of cancer cells of the stomach or colon which comprises:
providing with the cells an effective amount of anthocyanidin selected from the group consisting of cyanidin, delphinidin, malvidin, pelargonidin, petunidin and mixtures thereof, in order to inhibit the proliferation of the cells.

2. The method of claim 1 wherein the anthocyanidin is selected from the group consisting of malvidin and pelargonidin.

3. The method of claim 2 wherein the cells are stomach tumor cells.

4. The method of claims 1 or 2 wherein the cells are in a mammal.

5. The method of claims 1 or 2 wherein the cells are in a mammal and the compounds are fed orally to the mammal.
